(19) United States
(12) Patent Application Publication
(10) Pub. No.: US 2024/0216191 A1
(43) Pub. Date: Jul. 4, 2024

(54) Dirty Diaper Bin with Sealer

(71) Applicant: Numair Fakir, Brea, CA (US)

(72) Inventor: Numair Fakir, Brea, CA (US)

(21) Appl. No.: 18/092,780

(22) Filed: Jan. 3, 2023

(51) Int. Cl. A61F 13/551; B65F 1/00; B65F 1/06

(52) U.S. Cl. CPC A61F 13/5512; B65F 1/0006; B65F 1/062; B65F 2210/167; B65F 2240/132

(57) ABSTRACT

The present invention relates to a novel dirty diaper disposal system wherein the process includes a bin that is able to receive the soiled diaper, double seal it, and drop it to the main compartment in a packaged form. The proposed structure provides various features which provide enhanced comfort, convenience, and reliable output to its user.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

The present invention relates to waste disposal, and more particularly, to systems and methods for sealing objects in packages formed. The invention is particularly well suited for disposing of odorous objects such as soiled diapers.

### Description of the Related Art

On occasions, one would like to dispose of odorous objects in a convenient manner. This is particularly true of soiled, disposable diapers. If the diapers are collected in a disposal container as a batch, the odor can become quite strong and offensive. Efforts have been made to contain the odor of these soiled diapers. For example, U.S. Pat. No. 5,147,055, entitled “Diaper Container” shows a diaper container that includes an activated charcoal filter to retain and absorb odors within the container. U.S. Pat. No. 4,427,110, entitled, “Apparatus and Method for Handling used Disposable Diapers,” shows a canister that has a seal insert that 10 uses radially disposed slits in it to allow the diaper to pass through it. Others have developed sophisticated valve systems to attempt to contain the odor. See, e.g., U.S. Pat. Nos. 5,651,231; 5,765,339; and 5,655,680.

Another popular approach to disposing of such diapers has been with a device for twisting flexible tubing upon itself to form a pouch about the diaper. See generally, U.S. Pat. No. 5,590,512, entitled, “Apparatus for Using Packs of Flexible Tubing in Packaging”; U.S. Pat. No. 5,813,200, entitled, “Packaging and Disposal System”; and U.S. Pat. No. 4,869,049 entitled, “Apparatus and Methods for Using Packs of Flexible Tubing in Packaging,”; all three of these patents are incorporated herein by reference for all purposes.

There are multiple solutions that have been presented in the prior art. However, these solutions are limited and restricted to their conventional architecture, and design and have considerable shortcomings which adversely affect the convenience with which they can be used. The prior systems have certain limitations including the design of assemblies which make them not suitable for every type of user. Moreover, the ease-of-use ability of these assemblies is also questionable.

Therefore, it would be advantageous to design a system for disposing of soiled diapers in sealed package form. A container assembly is disclosed where the diaper is sealed once disposed of and is dropped in the main compartment. The proposed assembly presents a fairly simple and economical design that seals each soiled diaper separately in form of discrete packages Such systems use non-complicated, easy-to-use, mechanical sealing and diaper disposal device.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of a soiled diaper bin with a sealing feature.

Another object of the present invention is to provide an improved form of arrangement wherein once the diaper is disposed of, is received in the first compartment which seals the diaper pale and cuts it, and drops it in the main body.

Another object of this invention is to provide a new and improved assembly for disposing of the soiled diaper is provided which includes placing the object in a bag and using a sealing means to seal the bag closed in an air-tight manner to form a sealed package.

It is an object of the invention to provide a complete and continuous seal thereby eliminating leakage of odors from formed sealed package.

It is also the objective of the invention to provide an assembly where a relatively simple mechanical design allows relatively inexpensive manufacture and yet is practical to a high degree in use.

Thus, it is the objective to provide a new and improved system of dirty diaper management with a sealing feature. Other aspects, advantages, and novel features of the present invention will become apparent from the detailed description of the invention when considered in conjunction with the accompanying drawings.

This Summary is provided merely for purpose of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to provide an improved assembly which a sealable waste disposal system and method that address the shortcomings of previous systems and methods.

According to an aspect of the present invention, a waste disposal system for placing objects particularly soiled diapers is disclosed in sealed packages, wherein the system includes a container body, base, a top lid, and an inner lid, plastic bag roller, and sealing means that seals the soiled diaper bag in a substantially air-tight manner wherein the sealing means includes two sealing members that are operable to travel between an open position to a closed (or sealing) position. Once the diaper bag is sealed is dropped into the main body and the upper compartment is ready to receive the next diaper.

According to another aspect of the present invention, a method for disposing of odorous waste objects particularly soiled diapers is provided that includes the steps of placing the odorous objects in a container having a container body, base, a top lid, an inner lid, and a sealing mechanism to allow for an individually sealed package to be formed with odorous objects therein. The process involves a user throwing the soiled diaper by opening the top lid in the first compartment. As the diaper is received the sealing mechanism double seals the diaper bag in an air-tight manner.

Once the diaper is sealed, the sealed package is dropped in the main body, wherein the first compartment is ready to receive the next diaper. According to another aspect of the present invention, a method for disposing of odorous objects is provided that includes placing the object in a bag and using a sealing means to seal the bag closed in an air-tight manner to form a package.

The sealing is preferably accomplished through thermal means or by ultrasound or other such techniques. Energy is provided to the system through an electrical cord going to a sealing unit; alternatively, batteries or another electrical power source may be used.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

### BRIEF DESCRIPTION OF DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 shows the multiple views of a dirty diaper bin incorporating the embodiments of the invention.

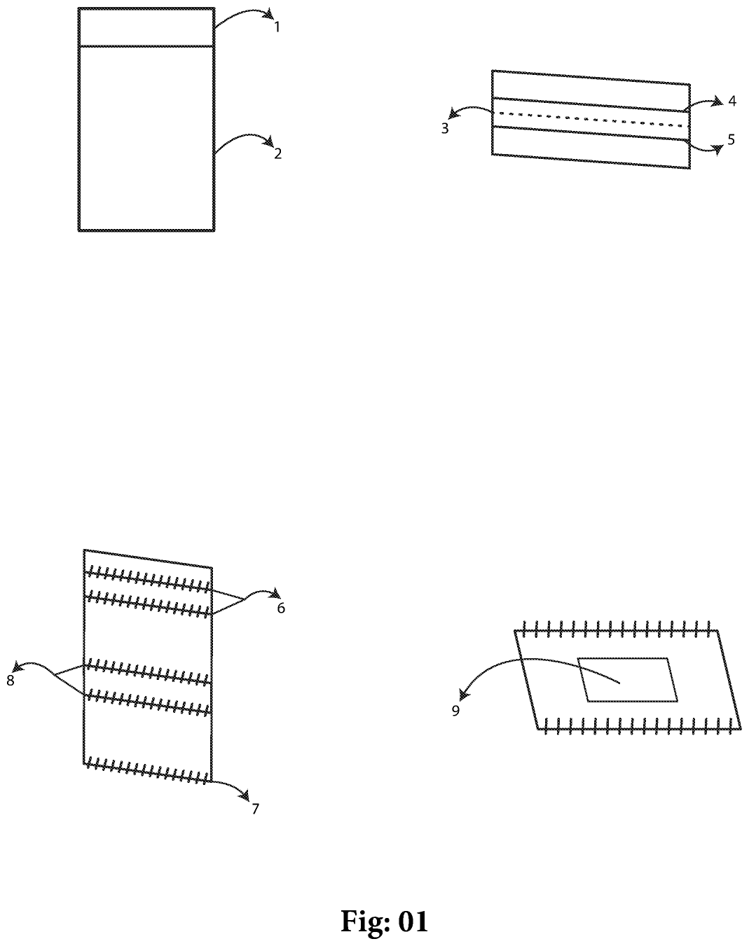

1. A soiled diaper disposal system wherein the improvement comprising:
Main body;
Top lid;
Sealer;
Plastic bags roller;
Base; and,
Energy source.
As per claim 1, a soiled diaper disposal system including a first compartment to receive a soiled diaper;
As per claim 1, a soiled diaper disposal system including a sealing mechanism to seal the diaper in a plastic bag;
As per claim 1, a soiled diaper disposal system including a double sealing mechanism to form an air-tight package; and,
As per claim 1, a soiled diaper disposal system including transferring the sealed package to the main body.
